(19) United States
(12) Patent Application Publication
(10) Pub. No.: US 2023/0143764 A1
(43) Pub. Date: May 11, 2023

(54) Guiding and Assessment System for Cervical Examination of Gravid Patients

(71) Applicant: Amy Gail Qualey, Atascadero, CA (US)

(72) Inventor: Amy Gail Qualey, Atascadero, CA (US)

(21) Appl. No.: 17/981,143

(22) Filed: Nov. 4, 2022

Related U.S. Application Data
(60) Provisional application No. 63/276,377, filed on Nov. 5, 2021.

(51) Int. Cl. A61B 5/00; A61B 5/107

(52) U.S. Cl. CPC A61B 5/435; A61B 5/1072; A61B 2560/0431

(57) ABSTRACT

The guiding and assessment system for cervical examination of gravid patients relates to devices, systems, and methods for use as educational and non-medical intervention facilitation tools for both medical professionals and gravid patients/persons having a baby to achieve a vaginal delivery through non-medical interventions such as position change, gravity positions, massage, pelvic mobility, etc. by assessing the presentation of the fetal head to the maternal pelvis, dilation, and effacement. To accomplish this, the system includes a portable device for measurement of cervical dilation, presentation of fetal position relative to the maternal pelvis, and/or measurement of effacement of the cervix in a gravid patient. The device includes semicircular open rings made of pliable material, as well as a plurality of dilation markings, a plurality of effacement markings, and a plurality of presentation markings, that represent measurements of cervical dilation, effacement and fetal head presentation.

### FIELD OF THE INVENTION

The present invention relates generally to the medical and surgical fields, namely, obstetrics and gynecology (OB/GYN) and the field of vaginal delivery. More specifically, the present invention relates to devices, systems, and methods used as facilitating and teaching tools for both medical professionals and gravid patients/persons having a baby to do a vaginal delivery through non-medical interventions such as position change, gravity positions, massage, pelvic mobility etc. by assessing presentation of fetal head to the maternal pelvis, dilation, and effacement.

### BACKGROUND OF THE INVENTION

Maternity care of gravid patients and childbirth have seen medical and technological advances that have drastically reduced maternal and infant mortality. According to the World Health Organization, while most pregnancies and births are uneventful, all pregnancies are at risk. Around 15% of all pregnant women will develop a potentially life-threatening complication that calls for skilled care, and some will require major obstetrical interventions to survive. In such cases, rapid assessment and decision-making based on clinical assessment and interventions with limited reliance on laboratory or other tests becomes paramount. These are interventions that, when used appropriately, can be life-saving procedures.

On the other hand, routine use of such interventions without valid indications, training, and proper facilitation and teaching systems, tools, and methods can transform childbirth from a normal physiologic process and family life event into a medical or surgical procedure. Every such intervention presents the possibility of untoward effects and additional risks that create the need for even more interventions with their own inherent risks. Unintended consequences to intrapartum interventions make it imperative to find alternative and functional apparatuses, devices, systems, and methods for use in facilitating and teaching both medical professionals and patients/persons having a baby to achieve a vaginal delivery through non-medical interventions such as position change, gravity positions, massage, pelvic mobility etc. by assessing the presentation of fetal head to maternal pelvis, dilation and effacement to promote natural childbirth processes and advocate for policies that focus on ensuring informed consent and alternative choices.

Some interventions and procedures have been routinely used in monitoring as well as managing complications in pregnancy and childbirth. The health care workers are generally taught to begin with a table that is often used by them to rapidly assess the woman's condition and initiate appropriate treatment, if so may be required. Every pregnancy has a risk of complications, and these complications, if they arise, may be harder to detect or treat without medical intervention. Thus, non-medical interventions form a shield and require proper training and facilitation of health care workers to allow natural and vaginal delivery. In order to provide health care workers with the information needed to differentiate between normal processes and a complication, and to educate and train them on description of normal labor and childbirth, guidance is often provided in the form of charts, diagrams, and tables but it remains an issue in terms of variance in training and understanding as a teaching and management tool so as to achieve vaginal delivery with minimal medical interventions.

This has identified a need for devices, systems, and methods for training, teaching, and facilitating health care workers/medical professionals, as well as educating the gravid patients/persons having a baby about the options to achieve a vaginal delivery through non-medical interventions such as position change, gravity positions, massage, pelvic mobility, etc. by assessing the presentation of the fetal head to the maternal pelvis, dilation and effacement, where the conventional methods and systems in place are lacking. But none of the conventional devices and methods resolve the aforementioned need in the art.

Conventionally, health care workers/medical practitioners use foam cutouts for the dilation, lifesavers for the effacement, and a head-and-pelvis for the presentation. But the systems and methods for the said crucial aspects of vaginal exams are not streamlined and it is cumbersome to consult conventional charts and tables and, also may lead to confusion and discomfort for the patients.

An objective of the present invention is to provide a system and methods to address the said need. It is an aim of the present invention to overcome the problems associated with conventional systems and methods by providing a portable device of the present invention as disclosed herein that can be carried in the pocket or on a badge and can help health care workers/medical practitioners to assess uniformly in cervical exams which are made streamlined as well as can be used to teach and educate the patients to understand what is happening to their cervix. The device of the present invention is further advantageous in that it can be used multiple times being made of material that is wipe-off-able and can be cleaned and reused in a sanitary manner, is small enough to carry in a portable manner, and instills confidence and uniformity among the health care workers/medical practitioners and alleviates the constant and tedious need to run out of the room to get confirmation on the exam.

### SUMMARY OF THE INVENTION

The present invention relates to devices, systems, and methods for use as educational and non-medical intervention facilitation tools for both medical professionals and gravid patients/persons having a baby to achieve a vaginal delivery through non-medical interventions such as position change, gravity positions, massage, pelvic mobility, etc. by assessing the presentation of the fetal head to the maternal pelvis, dilation, and effacement. The present invention provides a portable device for measurement of cervical dilation, presentation of fetal position relative to the maternal pelvis, and/or measurement of effacement of the cervix in a gravid patient. The device comprises semicircle open rings made of pliable material, wherein the semicircle open rings are numbered and measured in a range of 1 cm to 10 cm to measure dilation. Further, the device comprises five concentric half-circle rings to measure effacement, wherein said concentric half-circle rings are separated by 0.5 cm each. Furthermore, the device provides the presentation of fetal position relative to the maternal pelvis as assessed by a health care worker in a gravid patient. To that end, four round circles representing the fetal head with suture lines felt up and down and side to side are provided on the device.

### BRIEF DESCRIPTION OF THE DRAWINGS

FIG. 1 is a representative illustration of a representative embodiment of a portable device for measurement of cervical dilation, presentation of fetal position relative to the maternal pelvis, and/or measurement of effacement of the cervix in a gravid patient, as disclosed in the present invention.

FIG. 2 is a representative illustration of the concept of the portable device as disclosed in the present invention.

FIG. 3 is a representative illustration of a fetal head showing the various areas including Lambdoid suture, Sagittal suture, Coronal suture, Frontal suture, Anterior fontanelle, and Posterior fontanelle.

FIG. 4 is a representative illustration of the various directions of the fetal head or presentation of fetal position relative to the maternal pelvis or tail bone or the various positions of the presentation showing Direct Occiput Anterior (DOA), Left Occiput Anterior (LOA), Left Occiput Lateral (LOL), Left Occiput Posterior (LOP), Direct Occiput Posterior (DOP), Right Occiput Posterior (ROP), Right Occiput Lateral (ROL), and Right Occiput Anterior (ROA).

FIG. 5 is a representative illustration of the various presentations of the fetus (represented as a cartoon fetus) as oriented against the background of the maternal pelvis in the background showing presentations Right Occiput Posterior (ROP), Left Occiput Posterior (LOP), Right Occiput Anterior (ROA), and Left Occiput Anterior (LOA), where R is the direction Right, L is the direction Left, O is Occiput or back of the head of the fetus, P is the Posterior and describes the back or direction toward the back of the body, and A is the Anterior and describes the front or direction toward the front of the body.

### DETAILED DESCRIPTION OF THE INVENTION

Detailed embodiments of the present invention are disclosed herein. However, it is to be understood that the disclosed embodiments are merely exemplary of the present invention, which may be embodied in various systems. Therefore, specific details disclosed herein are not to be interpreted as limiting, but rather as a basis for teaching one skilled in the art to variously practice the present invention.

All illustrations of the drawings are for the purpose of describing selected versions of the present invention and are not intended to limit the scope of the present invention.

The present invention addresses the problem in the medical OB/GYN field of vaginal delivery in a gravid patient as discussed hereinabove by means of the references mentioned hereinabove and incorporated by reference.

The present invention provides devices, systems with guides for teaching, understanding, and facilitating natural vaginal birth with the intervention of cervical exams, and the method for health care workers/medical practitioners to perform such cervical exams and to educate the gravid patient/person giving birth to make informed decisions on childbirth and alternate options available to them to choose from to understand complications, if any, and informed decisions to tackle such complications based on real-time analysis and assessment using non-medical interventions. The present invention aims to offer a teaching tool and guide using the disclosed device and method to help everyone to use the same language and the same measurements for exams for uniformity and better and quicker decisions. The present invention is advantageous in that, a health care worker/medical practitioner can carry the disclosed piece of equipment/device since it is portable, and it can be used after a digital exam.

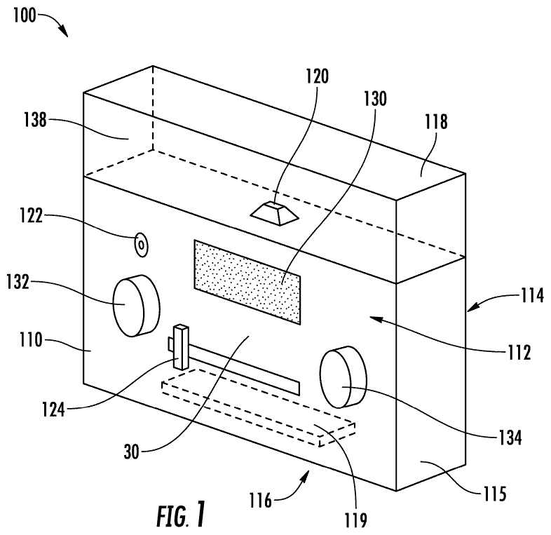

The following description is in reference to FIG. 1 through FIG. 5. According to a preferred embodiment, the present invention is a guiding and assessment system for cervical examination of gravid patients, wherein the system comprises a measurement panel 1, a central aperture 2, a plurality of arcs 3, a plurality of dilation markings 4, a plurality of effacement markings 5, and a plurality of presentation markings 6. Preferably, the measurement panel 1 is made of a pliable material, such as plastic, rubber, silicone, TCU etc., that may be easily wiped or cleaned. This is so that, the device of the present invention may be used multiple times for a single patient as well as on multiple patients and being made of a material that is wipe-off-able and cleanable helps in reusing the device in a sanitary manner. Further, it is preferred that the measurement panel 1 is small enough to carry in a portable manner. This is so that it instills confidence and uniformity among the health care workers/medical practitioners and alleviates the constant and tedious need to run out of the room to get confirmation on the exam. However, the measurement panel 1 may comprise any other material, size, components, arrangement of components etc. that are known to one of ordinary skill in the art, as long as the intents of the present invention are not altered.

As seen in FIG. 1, the measurement panel 1 comprises an arc region 1a and a base region 1b. Preferably, the central aperture 2 is a focal point, that is positioned centrally along the arc region 1a and the plurality of arcs 3 radiates from the central aperture 2. More specifically, the central aperture 2 is a central hole that delineates a first arc 3a. Further, the central aperture 2 may be used for holding the apparatus by inserting a finger through the aperture or may be used for hanging the device from a hook. To that end, the central aperture 2 traverses through the measurement panel 1. The plurality of arcs 3 is arranged around the central aperture 2, wherein a first diameter 7a of the first corresponding arc 3a that is adjacent to the central aperture 2 is less than a second diameter 7b of a second corresponding arc 3b, that is farther from the central aperture 2. Further, the plurality of arcs 3 is evenly distributed within the arc region 1a. More specifically, the plurality of arcs 3 are concentric half-circles wherein diameter of each of the plurality of arcs 3 ranges from 1 cm to 10 cm. For example, a difference in diameter between two adjacent arcs is 1 cm. Furthermore, a number of plurality of arcs 3 is 10. This is so that, the device may provide a realistic representation of dilation of the cervix as assessed by a health care worker in a gravid patient.

As seen in FIG. 1, the plurality of dilation markings is positioned along a first region 8a of the measurement panel 1. Preferably, the first region 8a constitutes a first half section of the arc region 1a, such as a left side section of the measurement panel 1. In the preferred embodiment, dilation may mean how open the cervix is, when measured in centimeters (cm). Conventionally, as medical professionals, their fingers are “trained” to know how far they spread and that is translated to how many cm is the cervical dilation in a gravid patient. To bring uniformity, the tool/device disclosed herein the present invention make sure that everyone is on the same page with the same dilation and it can further be used as a teaching tool to train the untrained health care workers/medical practitioners as well as to aid the understanding of the gravid patient/person giving birth to make informed decisions. In the disclosure of the present invention in the device, the diameter (part measured in cm) of the plurality of arcs 3 ranges from 1 cm to 10 cm and correlates with dilation measured in cm, as shown in FIG. 1 of the present disclosure. In other words, the plurality of dilation markings 4 is numbered and measured in a range of 1 cm to 10 cm. Preferably, the plurality of dilation markings 4 are superimposed on the measurement panel 1. However, the plurality of dilation markings 4 may be attached to the measurement panel 1 through any other techniques, such as sticking, painting, engraving etc. as long as the intents of the present invention are not altered. Furthermore, the plurality of dilation markings 4 is positioned along a first base line 9a created by a first set of terminal ends of the plurality of arcs 3.

It is an aim of the present invention to provide a multifunctional device or guiding tool for health care workers dealing with gravid patients. To that end, the plurality of effacement markings 5 is positioned along a second region 8b of the measurement panel 1, wherein the second region 8b is opposite to the first region 8a across the central aperture 2. In other words, the second region 8b constitutes a second half section of the measurement panel 1, such as a right-side section, that is opposite to the first region 8a. As used here, effacement means the thickness of the cervix. Generally, the cervix needs to get thinner (seen as a larger number of percentages of effacement) before it can dilate (stretch open). Effacement is usually measured in percentages wherein the unit of measurement is 0.5 cm and each 0.5 cm equals 25% effaced. In the disclosure of the present invention in the device, on one side of the device such as the right side of the device, as shown in FIG. 1, of the present disclosure, shows percentage (%) effacement measured in terms of concentric rings 0.5 cm apart, where each 0.5 cm rings denote a 25% of effacement. In other words, each of the plurality of effacement markings 5 is separated by 0.5 cm. In the preferred embodiment, the plurality of effacement markings 5 is positioned along a second base line 9b created by a second set of terminal ends of the plurality of arcs 3 opposite to the plurality of dilation markings 4.

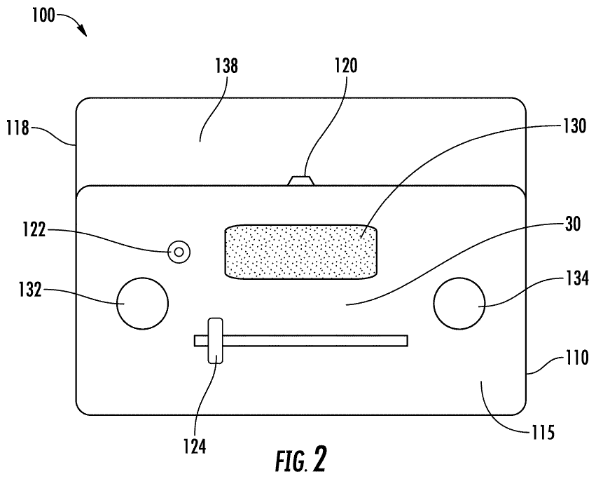

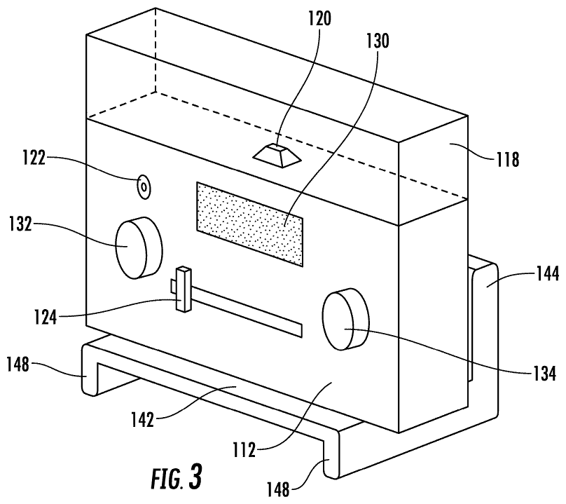

Continuing with the preferred embodiment, the plurality of presentation markings 6 is positioned within the base region 1b. Preferably, the base region 1b is the lower section of the measurement panel 1 that is positioned below the plurality of arcs 3. As used here, the plurality of presentation markings 6 or presentation may mean as to which way the fetal head is directed when compared to the maternal pelvis. This gives the health care worker/medical practitioner the required information about where the head is and how to rotate the maternal pelvis to get the fetal head to move down in the maternal pelvis. In the disclosure of the present invention in the device as shown in FIG. 1 and FIG. 2, at the base of the device, there are 4 circles that represent the fetal head as felt in an exam. More specifically, the plurality of presentation markings is positioned adjacent to the first baseline 9a and the second baseline 9b, opposite to the plurality of arcs 3.

The measurement of the presentation of the fetal head is in letters labeled as Occiput Posterior referred to as OP, Left Occiput Anterior referred to as LOT, Right Occiput Anterior referred to as ROT, and Occiput Anterior referred to as OA. The letters are representative of where the back (occipital) of the head is compared to the maternal pelvis. For example, the back of the fetal head is to the mother's tailbone is measured as OP or occiput posterior. The suture line would be felt like a vertical line. If the suture line is horizontal, the fetus could be ROT or LOT depending on if the back of the head is to the maternal left or right. As seen in FIG. 1, in the preferred embodiment, the plurality of presentation markings 6 is evenly distributed along a length of the base region 1b. Preferably, the plurality of effacement markings 5 and the plurality of presentation markings 6 are superimposed on the measurement panel 1. However, the plurality of effacement markings 5 and the plurality of presentation markings 6 may be attached to the measurement panel 1 through any other techniques, such as sticking, painting, engraving etc. as long as the intents of the present invention are not altered.

A more detailed description of the physical features of the present invention follows. According to the preferred embodiment, and in reference to FIG. 1, the present invention comprises a plurality of gaps 10. Preferably, the plurality of gaps 10 traverses through the measurement panel 1, and the plurality of gaps 10 is evenly distributed between the plurality of arcs 3. More specifically, each of the plurality of gaps 10 is positioned between the first corresponding arc 3a and the second corresponding arc 3b, wherein the first corresponding arc 3a and the second corresponding arc 3b are adjacent arcs from the plurality of arcs 3. In other words, the plurality of gaps 10 delineates the plurality of arcs 3, and each of the plurality of gaps 10 separate one arc from the other.

In the preferred embodiment, the present invention further comprises an extended region 11, and an access hole 12. Preferably, the extended region 11 laterally extends from a terminal edge 1c of the measurement panel 1, and the access hole 12 traverses through the extended region 11. As seen in FIG. 1, the extended region 11 is a U-shaped segment extending from the base region 1b of the measurement panel 1, and the access hole 12 is circular in shape. However, the extended region 11 and the access hole 12 may comprise any other shape, size, components, arrangement of components etc., as long as the intents of the present invention are not altered. In other words, the access hole 12 may either be in the middle or on the left base of the device for a way of attaching it to the thumb or for a method of hanging.

A detailed description of a preferred method of operation of the present invention follows. In an embodiment of the present invention, the method for measurement of cervical dilation, presentation of fetal position relative to the maternal pelvis, and/or measurement of effacement of the cervix in a gravid patient by conducting an exam is disclosed, the method comprising the steps of:

obtaining consent for the exam in the gravid patient after rationalizing and explaining the reason for the exam by a health care worker to the gravid patient;

laying the gravid patient flat on a surface with a roll or bump under the hips of the gravid patient;

explaining and assisting the gravid patient into position with heals together and knees apart by the health care worker;

obtaining consent from the gravid patient again to touch the vaginal area;

performing an examination of the vaginal area of the gravid patient by the health care worker for the measurement of cervical dilation, presentation of fetal position relative to the maternal pelvis, and/or measurement of effacement of the cervix in a gravid patient,

wherein the health care worker examines the vaginal area of the gravid patient by first using a gloved non-dominant hand to spread the labia of the gravid patient, then the health care worker gently while using a gloved dominant hand and doused in generous lubrication to insert a pointer finger and a middle finger of said dominant hand in the vagina of the gravid patient, wherein after entering the vagina of the gravid patient, the health care worker makes a peace sign of about 3 cm to 4 cm and feels for the anterior aspect of the vaginal wall and follows the wall backward while allowing the pointer finger and the middle finger to find the cervix and insert them into the cervix of the gravid patient to perform the exam for measurement of the distance of separation between the pointer finger and the middle finger at resting tone, and wherein subsequent measurements are done of the distance of separation between the pointer finger and the middle finger, and said measurements of the distance of separation between the pointer finger and the middle finger are seen as an increase in the measured diameter of the concentric half-circle rings of the device used as a measurement reference of the stretch of the cervix representing cervical dilation in the gravid patient said measured diameter of the concentric half-circle rings of the device providing the measurement of the cervical dilation.

Accordingly, the guiding and assessment system for cervical examination of gravid patients comprises monitoring how far an examiner's fingers can spread during examination of the gravid patient's cervix and analyzing that information to determine an estimate length to that analysis. Further, the estimate length is compared with the plurality of dilation markings 4 on the measurement panel 1 to assess an accurate dilation value in centimeters. In other words, the health care worker/medical professional measures the stretch of the cervix with their pointer finger and middle finger as per the conventional practice in the field, where the distance by which the said fingers separate indicates how dilated the cervix of the gravid patient is at a given time. Then the health care worker/medical professional takes the separated pointer finger and middle finger at the above-mentioned distance between them and compares it to the reference concentric half-circle rings on the disclosed device of the present invention, the concept is illustrated in

FIG. 1 and an embodiment of a prototype is illustrated in FIG. 2 of the present invention. Thus, the device as disclosed herein the present invention provides and functions as a measurement reference for the distance between the above-mentioned fingers to obtain the dilation of the cervix in the said gravid patient within a range of 1 cm to 10 cm as measured at the radius (base) of the said device. The plurality of arcs 3 stretch as a normal cervix would so it makes the device amenable to work as a better reference for measurement as discussed above. As disclosed in the method of the present invention, the correct dilation measurement technique is to measure the distance of separation between the above-mentioned fingers as a measure of dilation of the cervix in a gravid patient at resting tone, but the stretchiness of the cervix is informative for the health care worker/medical professional that it wants to or is ready to dilate further. The units of measurement as discussed above are cm of the distance between the above-mentioned fingers as compared to the reference on the measurement device of the present invention, where the device of the present invention is used as a measuring reference once the fingers are out of the vagina of the gravid patient. It is measured by inserting the pointer finger and middle finger of the dominant hand in the vagina, feeling for the cervix (imagine a donut), and inserting both fingers in the cervical os (hole). The cervical os is the opening in the cervix at each end of the endocervical canal. It would be understood by a health care worker/medical professional that when the fingers separate to see how dilated the cervix is, sometimes the cervix is accidentally stretched (meaning it is ready to dilate). When the fingers find the os they open like a peace sign. Health care workers/medical practitioners need to train their fingers to the amount open (peace sign) to know what the dilation is. They can measure the openness of their fingers to the radius of the half circles to confirm the cm of dilation. In other words, measured diameter of the plurality of arcs, or concentric half-circle rings of the device provides the measurement of the cervical dilation,

Similarly, in order to measure effacement, the system comprises, monitoring a thickness of the gravid patient's cervix during examination of the gravid patient's cervix and analyzing that information to determine an estimate thickness to that analysis. Further, the estimate thickness is compared to the plurality of effacement markings 5 on the measurement panel 1 to assess an accurate effacement value in percentages. As shown in FIG. 1 of the present disclosure, percentage (%) effacement is measured in terms of concentric rings or the plurality of arcs that are 0.5 cm in width, where each 0.5 cm rings denote a 25% of effacement. In other words, as shown in FIG. 1 and FIG. 2 of the present invention as disclosed here, the thickness of the cervix is the effacement, and it is measured in percentage or percent (%). The thinner the cervix, the higher the percent (ex 100% effacement is paper-thin, 0% effacement is 2 cm thick). The thickness of the cervix is related to the cervix dilating. The health care worker/medical practitioner can be taught using the device as disclosed herein the present invention by imagining and equating the cervix to a thick (0% effaced) rubber band. When contractions put pressure on the rubber band it is very difficult to open (dilate). Here, the cervix as the rubber band is imagined as being thinner (75% effaced or 0.5 cm thick). Contractions put pressure on a thin rubber band and the band will stretch (dilate). Effacement is measured while measuring dilation. As the health care worker/medical practitioner makes a peace sign for dilation and remembers how that felt, you also remember how thick the cervical os (donut) was on your finger.

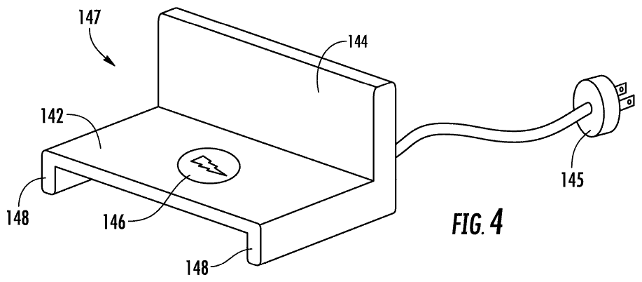

Furthermore, the system may comprise the steps of monitoring an orientation of a suture line of a fetal head through the cervix, during examination of the gravid patient's cervix. This is followed by comparing the suture line's orientation with the plurality of presentation markings to assess which way the fetal head is directed along a maternal pelvis of the gravid patient. More specifically, the presentation may mean as to which way the fetal head is directed when compared to the maternal pelvis. This gives the health care worker/medical practitioner the required information about where the head is and how to rotate the maternal pelvis to get the fetal head to move down in the maternal pelvis. It is important to understand and be able to assess and examine, the direction of the fetal head compared to the maternal pelvis to verify the direction of the fetal head or presentation of the fetal position relative to the maternal pelvis. An illustrative example of a fetal head is provided herein, in FIG. 3 of the disclosure of the present invention showing the various areas including Lambdoid suture, Sagittal suture, Coronal suture, Frontal suture, Anterior fontanelle, and Posterior fontanelle. The various directions of the fetal head or presentation of fetal position relative to the maternal pelvis or tail bone or the various positions of the presentation showing Direct Occiput Anterior (DOA), Left Occiput Anterior (LOA), Left Occiput Lateral (LOL), Left Occiput Posterior (LOP), Direct Occiput Posterior (DOP), Right Occiput Posterior (ROP), Right Occiput Lateral (ROL), and Right Occiput Anterior (ROA) as shown in a representative illustration in FIG. 4 of the disclosure of the present invention.

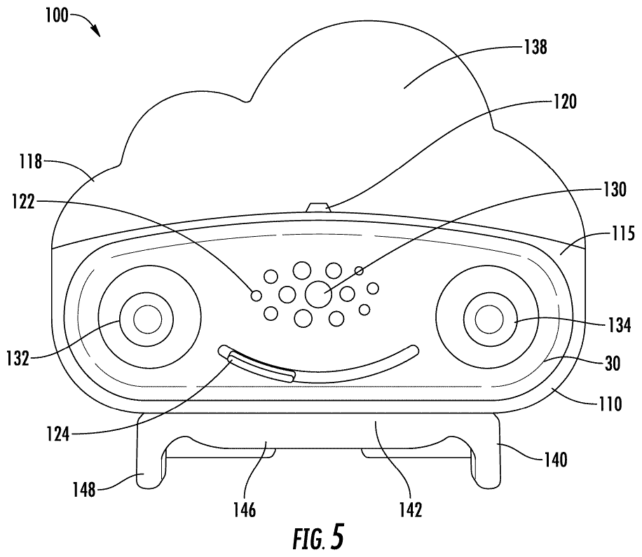

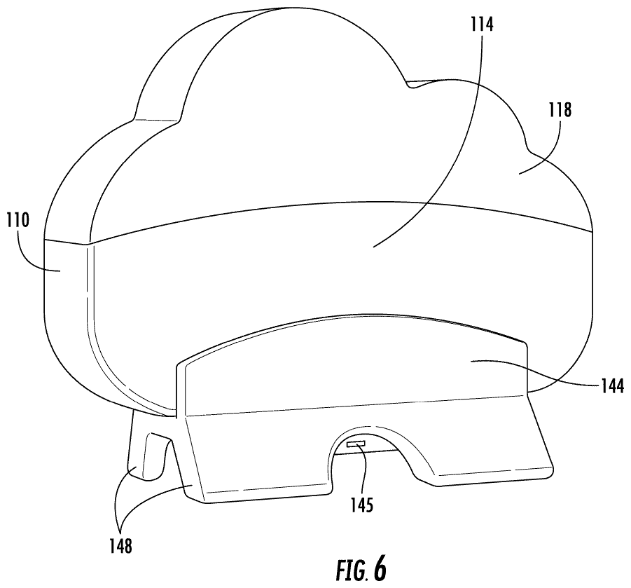

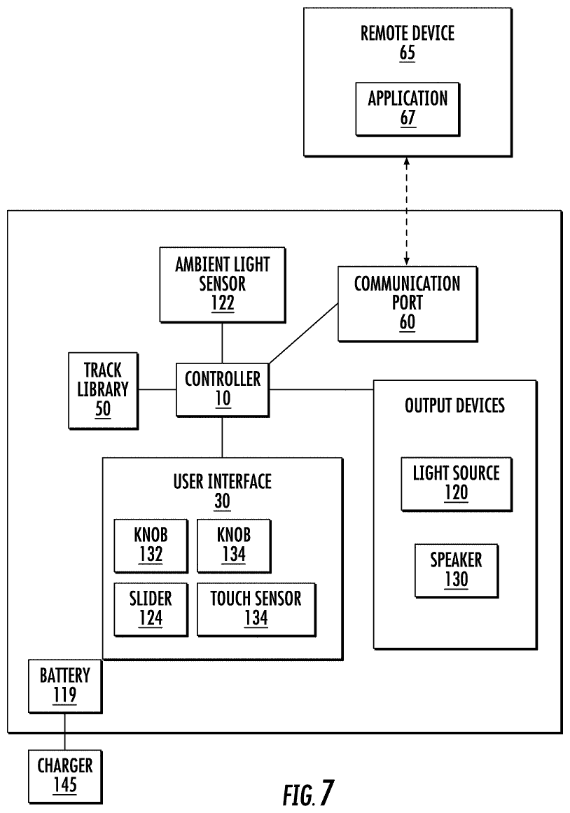

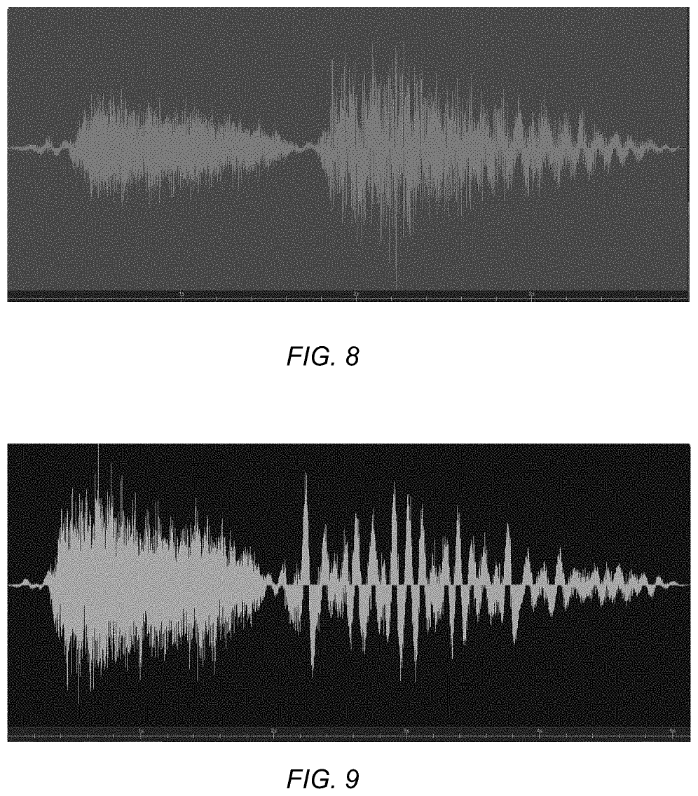

When assessing and examining the fetal presentation, for the health care worker/medical practitioner, this is an upside-down and backward concept. This is done to see what position (or the way the head is facing) compared to the maternal pelvis as shown in a representative illustration in FIG. 5 of the disclosure of the present invention. This can tell us if the head is crooked (asynclitic) or facing up (OP) or facing down (OA). Is the head centered in the cervix and pelvis or is it off to one side or the other? This helps the health care worker/medical practitioner to decide which way the birthing person needs to move to rotate the baby into position. The fetus will always turn towards the heavy part of the fetal body. For example, if the fetus is on the maternal right, the back of the head facing maternal right (ROT) and it may be required of the health care worker/medical practitioner to move the baby to face down (OA, most optimal way to deliver) and they can move the gravid patient/birthing person to a hands-and-knees position to get the heavy part of the fetus to rotate so that the fetal spine is closer to the maternal belly button. Presentation is measured while measuring the peace sign of dilation and the thickness of effacement. It is measured by feeling for the suture lines of the fetal skull. Is it up and down or side to side or centered or off to the side? What else does the health care worker/medical practitioner feel? Is there a diamond-shaped fontanel (anterior fontanelle) or a triangle shape fontanel (posterior fontanelle)?

In summary, in an embodiment of the present invention, a system with educational tools and a guide using the disclosed information received as dilation, effacement, and fetal presentation in the gravid patient using the disclosed portable device and method for measurement of cervical dilation, presentation of fetal position relative to the maternal pelvis, and/or measurement of effacement of the cervix in a gravid patient by conducting an exam to be used by medical professionals to reposition and evaluate labor progress, to help prevent the arrest of labor by actively managing cervical exams and using information gathered to rotate the presentation of the fetal head to the optimal position needed for pressure on the cervix to dilate and efface effectively and is used in shared decision making with patients to discuss labor progress and non-medical interventions such as a movement that can rotate a fetus into optimal position for effective dilation and effacement for vaginal delivery.

The present invention provides an educational tool for measuring cervical dilation comprising: semicircle open rings made of pliable material numbered and measured in a range of from 1 cm to 10 cm; a solid pliable piece of material with concentric half-circles where their diameter measured from distal sides and is in a range of from 1 cm to 10 cm; realistic representation of dilation whereas cervix can stretch but needs to be measured in resting tone, the concentric rings can stretch, but will be accurately measured in resting tone; an educational tool for medical professionals to gauge, measure, and train for cervical dilation exam; and an educational tool for use to teach the explanation of what exam means and next steps to the patients and/or the persons receiving the cervical dilation exam.

The present invention further provides a method of verification as to the direction of the fetal head or presentation of fetal position relative to the maternal pelvis compromising: four round circles representing the fetal head with suture lines felt up and down and side to side as defined as OA, OP, LOT, ROT, wherein the position of the fetal head compared to the maternal pelvis as recognized as suture lines represented by four circles labeled as OA, OP, LOT, ROT, wherein the information pertinent to optimal position of the fetal head compared to the maternal pelvis is obtained for successful vaginal delivery, and if the fetal head is not in an optimal position, such exam and method will give information as to next steps needed; provides a guide to repositioning patient in an optimal position, increasing pelvic mobility, for fetal head alignment compared to maternal pelvis for successful vaginal delivery; provides an educational tool for medical professionals to recognize the suture lines compared to the maternal pelvis to determine fetal presentation; and provides an educational tool used to teach the patient the fetal presentation compared to the maternal pelvis and the necessary movements required to be made to rotate the fetal presentation to an optimal position for vaginal delivery.

The present invention also provides a method of verification of measurement of effacement of cervix compromising: units of measurement of 0.5 cm which is equal to an effacement of 25%; concentric rings separated by 0.5 cm that are used to measure effacement by 25% increments, wherein larger the number, the thinner the cervix and greater the effacement; provides a guide to labor progress as to the strength and frequency of uterine contractions using the fetal head as presenting part putting pressure on the cervix to efface the cervix; provides an educational tool for medical professionals to measure thickness of the cervix as represented by percent effacement; and provides an educational tool used to teach the patient the percent effacement they are compared to the percent effacement needed to successfully deliver an infant vaginally.

Additionally, the present invention provides a guide using information received as dilation, effacement and fetal presentation compromising: educational tools used by medical professionals to reposition and evaluate labor progress; a tool used to help prevent the arrest of labor by actively managing cervical exams and using information gathered to rotate the presentation of the fetal head to an optimal position needed for pressure on the cervix to dilate and efface effectively; an educational tool used in shared decision making with patients to discuss labor progress and non-medical interventions such as a movement that can rotate a fetus into an optimal position for effective dilation and effacement for vaginal delivery. Further, the present invention provides a new and improved method which is a reliable system for multiple users to maintain uniform verbiage and units of measurement.

The target audience is anyone in the childbirth community. Nurses, doulas, midwives, doctors, and patients or persons having babies. This tool can encourage non-medical interventions that can change the outcome of the childbirth experience. The present disclosure is applicable for all the age groups, targeting all types of people.

While a specific embodiment has been shown and described, many variations are possible. With time, additional features may be employed. The particular shape or configuration of the platform or the interior configuration may be changed to suit the system or equipment with which it is used.

Although the invention has been explained in relation to its preferred embodiment, it is to be understood that many other possible modifications and variations can be made without departing from the spirit and scope of the invention as hereinafter claimed.

What is claimed is:

1. A guiding and assessment system for cervical examination of gravid patients, the system comprising:
a measurement panel;
a central aperture;
a plurality of arcs;
a plurality of dilation markings;
a plurality of effacement markings;
a plurality of presentation markings;
the measurement panel comprising an arc region and a base region;
the central aperture traversing into the measurement panel;
the central aperture being positioned centrally along the arc region;
the plurality of arcs radiating from the central aperture;
the plurality of arcs being arranged around the central aperture, wherein a first diameter of a first arc that is adjacent to the central aperture is less than a second diameter of a second arc, that is farther from the central aperture;
the plurality of arcs being evenly distributed within the arc region;
the plurality of dilation markings being positioned along a first region of the measurement panel;
the plurality of effacement markings being positioned along a second region of the measurement panel, wherein the second region is opposite to the first region across the central aperture; and
the plurality of presentation markings being positioned within the base region.

2. The guiding and assessment system for cervical examination of gravid patients of claim 1, wherein a diameter of a first arc adjacent to the central aperture is 1 cm.

3. The guiding and assessment system for cervical examination of gravid patients of claim 1, wherein a difference in diameter between two adjacent arcs is 1 cm.

4. The guiding and assessment system for cervical examination of gravid patients of claim 1, wherein a number of plurality of arcs is 10.

5. The guiding and assessment system for cervical examination of gravid patients of claim 1, wherein the plurality of dilation markings is numbered and measured in a range of 1 cm to 10 cm.

6. The guiding and assessment system for cervical examination of gravid patients of claim 1, wherein each of the plurality of effacement markings is separated by 0.5 cm.

7. The guiding and assessment system for cervical examination of gravid patients of claim 1, wherein the plurality of presentation markings is evenly distributed along a length of the base region.

8. The guiding and assessment system for cervical examination of gravid patients of claim 1, comprising:
a plurality of gaps;
the plurality of gaps traversing through the measurement panel;
the plurality of gaps being evenly distributed between the plurality of arcs; and
each of the plurality of gaps being positioned between a first corresponding arc and a second corresponding arc, wherein the first corresponding arc and the second corresponding arc are adjacent arcs from the plurality of arcs.

9. The guiding and assessment system for cervical examination of gravid patients of claim 8, wherein each of the plurality of gaps is 0.5 cm in width.

10. The guiding and assessment system for cervical examination of gravid patients of claim 1, comprising:
an extended region;
an access hole;
the extended region laterally extending from a terminal edge of the measurement panel; and
the access hole traversing through the extended region.

11. The guiding and assessment system for cervical examination of gravid patients of claim 1, wherein the plurality of dilation markings is positioned along a first base line created by a first set of terminal ends of the plurality of arcs.

12. The guiding and assessment system for cervical examination of gravid patients of claim 11, wherein the plurality of effacement markings is positioned along a second base line created by a second set of terminal ends of the plurality of arcs opposite to the plurality of dilation markings.

13. The guiding and assessment system for cervical examination of gravid patients of claim 11, wherein the plurality of presentation markings being positioned adjacent to the first baseline and the second baseline, opposite to the plurality of arcs.

14. The guiding and assessment system for cervical examination of gravid patients of claim 1, the system comprising:
monitoring how far an examiner's fingers can spread during examination of the gravid patient's cervix;
analyzing that information and determining an estimate length to that analysis; and
comparing the estimate length to the plurality of dilation markings on the measurement panel to assess a cervical dilation value in centimeters.

15. The guiding and assessment system for cervical examination of gravid patients of claim 1, the system comprising:
monitoring a thickness of the gravid patient's cervix during examination of the gravid patient's cervix;
analyzing that information and determining an estimate thickness to that analysis; and
comparing the estimate thickness to the plurality of effacement markings on the measurement panel to assess an accurate effacement value in percentages.

16. The guiding and assessment system for cervical examination of gravid patients of claim 1, the system comprising:
monitoring an orientation of a suture line of a fetal head through the cervix, during examination of the gravid patient's cervix; and
comparing the suture line's orientation to the plurality of presentation markings to assess which way the fetal head is directed along a maternal pelvis of the gravid patient.

17. A guiding and assessment system for cervical examination of gravid patients, the system comprising:
a measurement panel;
a central aperture;
a plurality of arcs;
a plurality of dilation markings;
a plurality of effacement markings;
a plurality of presentation markings;
a plurality of gaps;
the measurement panel comprising an arc region and a base region;
the central aperture traversing into the measurement panel;
the central aperture being positioned centrally along the arc region;
the plurality of arcs radiating from the central aperture;
the plurality of arcs being arranged around the central aperture, wherein a first diameter of a first arc that is adjacent to the central aperture is less than a second diameter of a second arc, that is farther from the central aperture;
the plurality of arcs being evenly distributed within the arc region;
the plurality of gaps traversing through the measurement panel;
the plurality of gaps being evenly distributed between the plurality of arcs;
each of the plurality of gaps being positioned between a first corresponding arc and a second corresponding arc, wherein the first corresponding arc and the second corresponding arc are adjacent arcs from the plurality of arcs;
the plurality of dilation markings being positioned along a first region of the measurement panel;
the plurality of effacement markings being positioned along a second region of the measurement panel, wherein the second region is opposite to the first region across the central aperture; and
the plurality of presentation markings being positioned within the base region.

18. The guiding and assessment system for cervical examination of gravid patients of claim 17, wherein the plurality of dilation markings is numbered and measured in a range of 1 cm to 10 cm.

19. The guiding and assessment system for cervical examination of gravid patients of claim 17, wherein each of the plurality of effacement markings is separated by 0.5 cm.

20. The guiding and assessment system for cervical examination of gravid patients of claim 17, wherein the plurality of presentation markings is evenly distributed along a length of the base region.
